(19) United States
(12) Patent Application Publication
(10) Pub. No.: US 2008/0187941 A1
(43) Pub. Date: Aug. 7, 2008

(54) METHOD FOR PREPARING FUSION PROTEIN BY TRANS-SPLICING METHOD

(75) Inventors: Masayuki Kawakami, Kanagawa (JP); Hiroshi Ueda, Tokyo (JP)

Correspondence Address: SUGHRUE MION, PLLC, 2100 PENNSYLVANIA AVENUE, N.W., SUITE 800, WASHINGTON, DC, 20037, US

(73) Assignees: FUJIFILM Corporation, Tokyo (JP); THE UNIVERSITY OF TOKYO, Tokyo (JP)

(21) Appl. No.: 11/832,369

(22) Filed: Aug. 1, 2007

(30) Foreign Application Priority Data
Feb. 2, 2007 (JP) 023974/2007

(51) Int. Cl. G01N 33/535; C12P 21/00

(52) U.S. Cl. 435/7.92; 435/69.7

(57) ABSTRACT

An object to be achieved by the present invention is to provide a method that enables convenient production of a target fusion protein within a short time without constructing any expression vector. The present invention provides a method for producing a fusion protein comprising a first protein and a second protein, which comprises the steps of: introducing a nucleic acid molecule (pre-trans-splicing molecule) or a vector capable of expressing such a nucleic acid molecule into a cell expressing the first protein, wherein the nucleic acid molecule contains a gene sequence encoding the second protein and a sequence which is capable of inducing trans-splicing through binding to pre-mRNA that is generated from a region containing a gene sequence that encodes the first protein; and recovering a fusion protein comprising the first protein and the second protein generated within the cell.

### TECHNICAL FIELD

The present invention relates to a method for producing fusion proteins by a trans-splicing method.

### BACKGROUND ART

Trans-splicing is a kind of splicing by which exons from separately transcribed precursor RNA molecules are joined when a mature messenger RNA is generated.

A gene on chromosomal DNA contains coding regions (exons) and is generally transcribed into a pre-mRNA containing intervening noncoding regions (introns). Such introns are removed from the pre-mRNA via a fine process referred to as splicing. Splicing is known to take place as an interaction coordinated by several small ribonucleoproteins (snRNPs) and many protein factors that assemble to form an enzyme complex known as a spliceosome.

Pre-mRNA splicing proceeds by a 2-step mechanism. The 1^{st} step involves cleavage of 5′splice site so as to generate a “free” 5′ exon and a lariat intermediate. The 2^{nd} step involves freeing introns in the form of lariat products, as the 5′ exon is ligated to 3′exon. These steps proceed via catalysis involving small nuclear ribonucleoproteins and a protein complex referred to as spliceosome. These splicing reaction sites are defined by consensus sequences in the peripheries of the 5′ and 3′splice sites. The 5′ splice site consensus sequence is AG/GURAGU (here, A=adenosine, U=uracil, G=guanine, C=cytosine, R=purine, and /=splice site). The 3′ splice region is composed of three individual sequential elements: a branch point or branch site, a polypyrimidine tract, and the 3′ consensus splice sequence (YAG). These elements roughly define the 3′ splice region. The 3′ splice region can contain a 100-nucleotide intron upstream of the 3′splice site. A consensus sequence of a mammalian branch point is CURAY (here, Y=pyrimidine). Underlined A is a branch formation site (BPA=branch point adenosine). The 3′ consensus splice sequence is YAG/G. A polypyrimidine tract is generally observed between a branch point and a splice site, which is important in a mammalian system for effective use of a branch point and recognition of the 3′ splice site. The first Y, A, and G (trinucleotides) located downstream of a branch point and a polypyrimidine tract forms a most-frequently-used 3′ splice site (Smith, C. W. et al., 1989, Nature 342: 243-247).

In most cases, the splicing reaction takes place within the same pre-mRNA molecule, which is referred to as cis-splicing. Splicing that takes place between two independently-transcribed pre-mRNAs is referred to as trans-splicing. Trans-splicing was discovered for the first time in Trypanosoma (Sutton & Boothroyd, 1986, Cell 47: 527; Murphy et al., 1986, Cell 47: 517) and then discovered in nematode, flatworm (Rajkovic et al., 1990, Proc. Natl. Acad. Sci. U.S.A., 87: 8879; and Davis et al., 1995, J. Biol. Chem. 270: 21813), and plant mitochondria (Malek et al., 1997, Proc. Natl. Acad. Sci. U.S.A. 94: 553).

A method that involves expressing a protein using such trans-splicing technique and then using the protein for gene therapy or protein identification methods and a method that involves applying the technique to imaging within animals have been reported to date (JP Patent Publication (Kohyo) No. 2004-525618 A; Gene therapy: Puttataju, M. et al, Nature Biotechnology, vol. 17, 246-252 (1999); and Imaging within animals: C. E. Walsh et al, Molecular Therapy, vol. 12, 1006-1012 (2005)).

Meanwhile, a method that has often been employed conventionally when a fusion protein of two or more proteins is prepared involves cloning a gene sequence encoding each target protein, constructing an expression vector into which such genes have been introduced, and then preparing the fusion protein by Escherichia coli or the like. However, such method requires complicated experimental protocols, including cloning of gene sequences encoding target proteins, construction of expression vectors, and the like. Accordingly, a new expression vector has been necessary for preparation of different types of fusion protein, even when the relevant proteins share a partially common portion.

### DISCLOSURE OF THE INVENTION

An object of the present invention is to solve the above problems of conventional techniques. Specifically, an object to be achieved by the present invention is to provide a method that enables convenient production of a target fusion protein within a short time without constructing any expression vectors. Moreover, an object to be achieved by the present invention is to provide a method for producing a fusion protein with high general versatility that enables production of proteins of the same type by the same method (reagent).

As a result of intensive studies to achieve the above objects, the present inventors have discovered that a target fusion protein can be produced conveniently within a short time by recovering fusion proteins generated within cells through the use of trans-splicing. Thus, the present inventors have completed the present invention.

Specifically, the following inventions are provided according to the present invention.

(1) A method for producing a fusion protein comprising a first protein and a second protein, which comprises the steps of:

introducing a nucleic acid molecule (pre-trans-splicing molecule) or a vector capable of expressing such a nucleic acid molecule into the cell expressing the first protein, wherein the nucleic acid molecule contains a gene sequence encoding the second protein and a sequence which is capable of inducing trans-splicing through binding to pre-mRNA that is generated from a region containing a gene sequence that encodes the first protein; and recovering a fusion protein comprising the first protein and the second protein generated within the cell.

(2) A method for producing a fusion protein comprising a first protein and a second protein, which comprises the steps of:

introducing a first nucleic acid molecule containing a gene sequence that encodes the first protein or a vector capable of expressing the nucleic acid molecule and a second nucleic acid molecule (pre-trans-splicing molecule) or a vector capable of expressing the nucleic acid molecule into a cell in which trans-splicing can take place, wherein the second nucleic acid molecule contains a gene sequence encoding the second protein and a sequence which is capable of inducing trans-splicing through binding to pre-mRNA that is generated from a region containing a gene sequence that encodes the first protein; and

recovering a fusion protein comprising the first protein and the second protein generated within the cell.

(3) The method according to (1) or (2), wherein the nucleic acid molecule (pre-trans-splicing molecule) containing a gene sequence encoding the second protein and a sequence which is capable of inducing trans-splicing through binding to pre-mRNA that is generated from a region containing a gene sequence that encodes the first protein is a nucleic acid molecule containing at least one sequence selected from among a 5′ splice sequence, a 3′ splice sequence, and an Sμ sequence or a vector capable of expressing the nucleic acid molecule.

(4) The method according to (1) or (2), wherein the nucleic acid molecule (pre-trans-splicing molecule) containing a gene sequence encoding the second protein and a sequence which is capable of inducing trans-splicing through binding to pre-mRNA that is generated from a region containing a gene sequence that encodes the first protein is a nucleic acid molecule containing:

(a) at least one sequence selected from among a 5′ splice sequence, a 3′ splice sequence, and an Sμ sequence;

(b) a branch point; and

(c) a pyrimidine tract;

or is a vector capable of expressing the nucleic acid molecule.

(5) The method according to any one of (1) to (4), wherein the nucleic acid molecule (pre-trans-splicing molecule) containing a gene sequence encoding the second protein and a sequence which is capable of inducing trans-splicing through binding to pre-mRNA that is generated from a region containing a gene sequence that encodes the first protein is a nucleic acid molecule encoded by a plasmid vector or a retrovirus vector.

(6) The method according to any one of (1) to (5), wherein the first protein is a protein containing an antibody H chain variable region protein (VH).

(7) The method according to any one of (1) to (6), wherein the first protein is a protein containing the antibody H chain variable region protein (VH) and the second protein is a protein containing an antibody H chain constant region CH1 protein.

(8) The method according to any one of (1) to (5), wherein the first protein is a protein containing an antibody L chain variable region protein (VL).

(9) The method according to any one of (1) to (5) or (8), wherein the first protein is a protein containing the antibody L chain variable region protein (VL) and the second protein is a protein containing an antibody L chain constant region CL protein.

(10) The method according to any one of (1) to (9), wherein the cell expressing the first protein is a cell producing an antibody.

(11) The method according to any one of (1) to (9), wherein the cell expressing the first protein is a hybridoma.

(12) The method according to any one of (1) to (9), wherein the cell expressing the first protein is a DT40 chicken somatic cell.

(13) The method according to any one of (1) to (12), wherein the second protein is a protein containing an enzyme.

(14) The method according to any one of (1) to (12), wherein the second protein is a protein containing alkaline phosphatase, peroxidase, β-galactosidase, or luciferase.

(15) An immunoassay method, comprising the steps of: producing a fusion protein by the method according to any one of (1) to (14); and performing immunoassay using the thus obtained fusion protein.

(16) An immunoassay method, comprising the steps of: producing a fusion protein containing an antibody H chain variable region protein (VH) and/or an antibody L chain variable region protein (VL) by the method according to any one of (1) to (14); and performing immunoassay using the thus obtained fusion protein.

In the present invention, expression vector construction is not required. Hence, a target fusion protein can be produced conveniently within a short time. According to the present invention, proteins of the same type can be produced by the same method (reagent). Hence, a method for producing a fusion protein with high general versatility is provided.

### BRIEF DESCRIPTION OF THE DRAWINGS

FIG. 1 shows the structure of pSV-Vμ1.

FIG. 2 shows the structure of pscFv-SEAP.

FIG. 3 shows the structures of 3 types of TS vector (pTS-3′ ss-SEAP, pTS-Sμ1-SEAP, and pTS-Sμ2-SEAP).

FIG. 4 shows the outline of a transfection experiment.

FIG. 5 shows the generation of mRNA through trans-splicing as confirmed by RT-PCR.

FIG. 6 shows the results of determining the alkaline phosphatase activity of the culture supernatants of transfected cells.

FIG. 7 shows an outline of trans-splicing.

### PREFERRED EMBODIMENTS OF THE INVENTION

### (1) Characteristics of the Present Invention

The present invention involves the use of trans-splicing as a method for expressing a fusion protein. The fusion protein of the present invention may be prepared by preparing fusion genes and then artificially binding two or more types of different proteins and is not particularly limited. Examples of such fusion protein include a fusion protein of an antibody variable region and an enzyme.

The present invention involves introducing a nucleic acid molecule (pre-trans-splicing molecule, PTM) or a vector capable of expressing the nucleic acid molecule into a cell expressing a first protein, wherein the nucleic acid molecule contains a gene sequence encoding the second protein and a sequence which is capable of inducing trans-splicing through binding to pre-mRNA that is generated from a region containing a gene sequence that encodes the first protein. Alternatively, according to another embodiment of the present invention: a first nucleic acid molecule (PTM) or a vector capable of expressing the nucleic acid molecule, wherein the first nucleic acid molecule contains a gene sequence encoding the second protein and a sequence which is capable of inducing trans-splicing through binding to pre-mRNA that is generated from a region containing a gene sequence that encodes the first protein, and a second nucleic acid molecule or a vector capable of expressing the nucleic acid molecule, wherein the second nucleic acid molecule contains a gene sequence encoding the first protein and a sequence which is capable of inducing trans-splicing through binding to the first nucleic acid molecule (PTM), are introduced into a cell in which trans-splicing can take place. Thus, intracellular trans-splicing is induced, so that a fusion protein comprising the first protein and the second protein is generated.

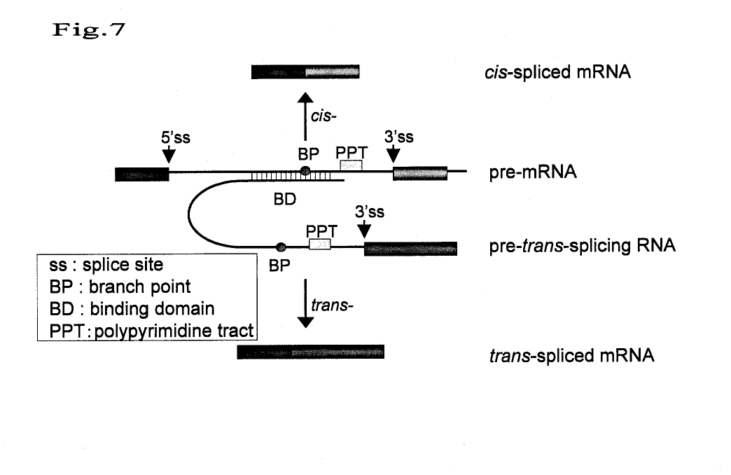

For induction of splicing, the 5′ splice site (5′ ss)/BP (branch point)/polypyrimidine tract (PPT)/3′ splice site (3′ ss) should be generally arranged in such order (towards the 5′ to 3′ direction) within an intron. Induction of trans-splicing requires the selection of any one sequence within a target intron, designing of a binding domain (BD) that is RNA capable of specifically binding to a selected sequence, and construction of a molecule (PTM) prepared by binding another set of BP/PPT/3′ss to BD. PTM is generally RNA that can be expressed via the use of vector DNA introduced into cells (FIG. 7).

Any binding domain (BD) sequence can be employed, as long as it is a sequence existing in an intron to which pre-RNA and PTM can bind. Specifically, the positional relationship of a binding domain (BD) sequence with other required components (5′ss/BP/PPT/3′ss) is not particularly limited. For example, a BP may be present or absent in a binding domain.

A target binding domain (BD) generally interacts with a 100-bp (or longer) sequence within an intron through complementary strand formation. In addition to this, 100 or more copies of a repeated sequence of tgagc or tgggg, which is referred to as an Sμ region, are present between the 5′ splice region and BP in the IgH V-Cμ intron used in the Example of the Description. Sμ is a sequence capable of inducing a class switch from IgM to another class with the help of cytidine deaminase, referred to as AID, which functions here. It is thought that Sμ becomes partially deleted because of a class switch reaction from IgM to IgG, IgA, or the like, and it is also thought that is often remains after it has been bound to another S region.

BD that generally has been used frequently is BD complementary to a BP, PPT, and 3′ss. This is because it can be expected that cis-splicing can be suppressed by masking of such portions via duplex formation thereof. However, since these sequences are consensus sequences that exist in every class II intron, the resulting reaction specificity may be lowered even though improvement in reaction efficiency is targeted. In the Example of the Description, the present inventors selected such Sμ region as a target sequence and succeeded in obtainment of trans-splicing efficiency at a level equivalent to that of TPM containing the above components used therein. The Sμ region is considered to have high specificity because it is present in only an antibody H chain, and it is considered to have high general versatility because it is also contained in introns of cells producing antibodies of any class. As described above, according to a preferred embodiment of the present invention, a target binding domain (BD) can contain the Sμ region. However, a BD sequence complementary to 3′ss is the same as those in other antibodies of the same (sub) class. Hence, such BD can be used as a BD that is specific to an antibody of a known (sub) class, including an L chain.

Recently, it has been reported that the length of an intron is an important element with respect to trans-splicing efficiency (Takahara et al., 2006, Mol. Cell, 18: 245-251). Hence, trans-splicing efficiency can be increased in the case of antibodies because the length of the intron between V and C is as long as several kilobases. Actually, a product (approximately 4% of human IgM derived from cis-splicing) thought to be derived from trans-splicing between a human V region and endogenous IgG1 has been observed in human antibody transgenic mice (Shimizu et al., 1989, Proc. Natl. Acad. Sci. U.S.A., 86: 8020-8023). Therefore, it is desired to use an intron between V and C as a target intron for trans-splicing in terms of efficiency. For example, when the purpose is to prepare a fusion protein containing a CH1 (or CL) such as a Fab-enzyme, it is desired that such CH1 (or CL) be contained in the second protein.

### (2) Pre-Trans-Splicing Molecule to be Used in the Present Invention

The present invention relates to a method for producing a fusion protein through trans-splicing. According to the method of the present invention, a pre-trans-splicing molecule (hereinafter, referred to as “PTM”) that is used herein is designed to interact with a target pre-mRNA molecule (hereinafter, referred to as “pre-mRNA”) and cause the formation of a chimeric RNA molecule, so as to mediate the trans-splicing reaction. The method of the present invention comprises causing the above-described PTM to come into contact with target pre-mRNA under conditions in which a portion of PTM is spliced into pre-mRNA so that a novel chimeric RNA is formed. Examples of target cells to be used herein include, but are not limited to, cells associated with immunity such as cells producing monoclonal antibodies (e.g., myeloma cells). In the present invention, hybridomas can be preferably used. A “hybridoma” is a cell obtained by cell fusion of a spleen B cell of an animal immunized with an antigen with a myeloma cell (myeloma). Hybridomas are cells possessing both the ability of B cells to produce antibodies and the ability of myelomas to undergo infinite proliferation. A homogeneous monoclonal antibody specific to a target antigen can be produced by the cloning of such hybridoma.

A pre-trans-splicing molecule (PTM) to be used in the present invention preferably contains: (a) a sequence containing at least one sequence selected from among a 5′ splice sequence, a 3′ splice sequence, and an Sμ sequence and being capable of binding to pre-mRNA generated from a region containing a gene sequence encoding a first protein; (b) a branch point; (c) a pyrimidine tract; and (d) a gene sequence encoding a second protein.

The method of the present invention involves causing the above PTM to come into contact with pre-mRNA under conditions in which a portion of PTM is trans-spliced to a portion of pre-mRNA, so as to generate a novel chimeric RNA. The pre-mRNA is expressed in a specific cell type, so as to enable targeted expression of a fusion protein in a selected cell type.

A target binding domain for PTM is complementary to the targeting region existing in the intron of the selected pre-mRNA and in an antisense orientation. Not only one binding domain, but also more than one binding domains may be contained. In the Description, a target binding domain is defined as any one sequence that imparts binding specificity and that is located close to and thus anchors pre-mRNA so that the nuclear spliceosome processing mechanism can perform trans-splicing of a portion of PTM to a portion of pre-mRNA. Such target binding domain can contain up to several thousand nucleotides. In a preferred embodiment of the present invention, such binding domain may contain at least 10 or 30 or up to several hundred nucleotides. As disclosed in JP Patent Publication (Kohyo) No. 2004-525618 A, the specificity of PTM can be enhanced by increasing the length of the target binding domain. For example, a target binding domain may contain several hundred or more nucleotides. Furthermore, a target binding domain may be “linear” and the RNA may be folded so as to form a secondary structure that can stabilize the complex and thus enhance splicing efficiency. When a plurality of binding domains are contained, a second target binding domain may be located on the 3′ end of the molecule and can be incorporated into PTM to be used in the present invention. Absolute complementarity is preferred, but it is not always required. A sequence “complementary” to a portion of RNA in the Description means a sequence that hybridizes to the RNA and has complementarity sufficient for stable double helix formation. Ability to undergo hybridization can depend on both the degree of complementarity and the length of a nucleic acid. In general, the longer the nucleic acid for hybridization, the more stable the formation of a double helix containing more nucleotide mispairings with RNA. Persons skilled in the art can confirm the tolerance of mispairing or length of a double helix by examining the stability of a complex that has undergone hybridization using a standard method.

A PTM molecule can contain a branch point, a pyrimidine tract, a 5′ splice sequence, and a 3′ splice sequence. Consensus sequences of such 5′ splice sequence and 3′ splice sequence to be used in RNA splicing are known in the art. Furthermore, altered consensus sequences that can function as such 5′ splice sequence and 3′ splice sequence can also be used in the present invention. The consensus 5′ splice site sequence is AG/GURAGU (here, A=adenosine, U=uracil, G=guanine, C=cytosine, R=purine, and /=splice site). The 3′ splice site may be composed of 3 different sequential elements: a branch point or branch site, a polypyrimidine tract, and the 3′ consensus sequence (YAG). The consensus sequence of a mammalian branch point is YNYURC (here, Y=pyrimidine). A polypyrimidine tract is located between a branch point and a splice site acceptor and is important in terms of the use of various branch points and recognition of the 3′ splice site.

PTM to be used in the present invention is designed so that a novel chimeric RNA is produced in target cells. For example, PTM to be used in the present invention may be in any form, including RNA molecules or DNA vectors to be transcribed into RNA molecules. The method of the present invention involves delivering PTM to a target cell and causing PTM to bind to pre-mRNA, so as to form chimeric RNA containing a portion of the PTM molecule, which is spliced to a portion of pre-mRNA.

A nucleic acid molecule to be used in the present invention may be RNA or DNA or a derivative or an altered product thereof. Such nucleic acid molecule may also be a single strand or a double strand. Such nucleic acid may also be composed of deoxyribonucleotide or ribonucleoside. Such nucleic acid may also be composed via phosphodiester bonds or other altered bonds. Another example of the term “nucleic acid” is a nucleic acid composed of nucleotides other than the 5 biologically observed types of nucleotide (adenine, guanine, thymine, cytosine, and uracil).

RNA and DNA molecules to be used in the present invention can be prepared by methods for DNA and RNA molecule synthesis known in the art. For example, a nucleic acid may be chemically synthesized using commercial reagents and a synthesizer according to a method known by persons skilled in the art (e.g., Gait, 1985, Oligonucleotide Synthesis: A Practical Approach, IRL Press, Oxford, England). Alternatively, an RNA molecule can also be prepared by in vitro and in vivo transcription of a DNA sequence encoding the RNA molecule. Such DNA sequence can be incorporated into various vectors in which an appropriate RNA polymerase promoter such as a T7 or SP6 polymerase promoter has been incorporated. RNA can be produced at high yields through in vitro transcription using a plasmid such as pSP65 (Promega Corp., Madison, Wis.). Furthermore, RNA can also be produced using an RNA amplification method such as Q-β amplification.

A base portion, a sugar portion, or a phosphate backbone of a nucleic acid molecule to be used herein can be altered in order to improve molecular stability, hybridization, and transport into cells, for example. For example, when PTM is altered so that the overall charge is lowered, incorporation of the molecule into cells can be improved. Furthermore, alterations to reduce sensitivity to nuclease degradation can also be performed. A nucleic acid molecule to be used herein may also contain a peptide (e.g., for targeting a host cell receptor in vivo) or another adjunctive group such as a cell membrane or an intercalating agent. For the same purpose, the nucleic acid molecule may be conjugated to another molecule such as a peptide, a hybridization-inducing crosslinking agent, a transport agent, or a hybridization-inducing cleavage agent. Various other types of known alteration of a nucleic acid molecule can be introduced as measures for elevating intracellular stability and half-life. A possible alteration is, but is not limited to, addition of a flanking sequence of ribo- or deoxynucleotide to the 5′ and/or 3′ end of the molecule. Under some environments in which it is desirable to increase stability, a nucleic acid having an altered intra-nucleoside bond such as 2′-O-methylation can be preferred. Such nucleic acid containing an altered intra-nucleoside bond can be synthesized using a reagent and a method known by persons skilled in the art.

A nucleic acid to be used herein can be purified by a method known in the art. For example, such nucleic acid can be purified by reverse phase chromatography or gel electrophoresis. The purification method differs to some extent depending on the size of the nucleic acid to be purified.

When a nucleic acid molecule encoding a trans-splicing molecule, synthetic PTM, is used, cloning of such nucleic acid molecule into an expression vector can be performed using a cloning technique known in the art. A method for recombinant DNA technology that is generally known in the art and can be used herein is described in Ausubel et al., (ed.), 1993, Current Protocols in Molecular Biology, John Wiley & Sons, NY; and Kriegler, 1990, Gene Transfer and Expression, A Laboratory Manual, Stockton Press, NY.

The use of DNA encoding target PTM enables the provision of large-scale DNA replication and recombination of various host vector systems containing essential elements that direct PTM transcription. For example, a vector can be introduced by causing cells to incorporate the vector, so as to be able to direct the transcription of the PTM molecule. Such vector may be maintained as an episome or incorporated into chromosome, as long as it is transcribed to produce target RNA. Such vector can be constructed by standard recombinant DNA technology in the art.

Vectors encoding target PTM may be in other forms known in the art, such as plasmids, viruses, or other forms, which are used for replication and expression of target PTM in mammalian cells. The expression of a sequence encoding PTM can be regulated using any promoter known in the art, so as to cause the sequence to function in mammalian (e.g., human) cells. Such promoter may be an inducible or constitutive promoter. Examples of such promoter include, but are not limited to, an SV40 early promoter region, a promoter contained in the 3′ long terminal repeat of Rous sarcoma virus or moloney-murine leukemia virus (MoMLV), a herpes thymidine kinase promoter, a regulatory sequence of a metallothionein gene, a CMV virus promoter, and a human chorionic gonadotropin-β promoter. A recombinant DNA construct that can be directly introduced into a tissue site can be prepared using any types of plasmid, cosmid, YAC, and viral vector. Alternatively, a viral vector that selectively infects a desired target cell can also be used. A vector that is preferably used in the present invention is a plasmid vector or a retrovirus vector.

Examples of various delivery systems that are known by persons skilled in the art and can be used in the present invention include encapsulation into liposomes, microparticles, microcapsules, recombinant cells capable of expressing a composition, endocytosis mediated by a receptor, construction of a nucleic acid as a portion of a retrovirus vector or another vector, DNA injection, electroporation, and transfection mediated by calcium phosphate.

In the present invention, a viral vector containing PTM can also be used. For example, a retrovirus vector that can be used herein has been altered so that a retrovirus sequence not required by viral genome packaging and incorporation into host cell DNA is deleted. Alternatively, an adenovirus or an adeno-associated virus vector can be used for gene delivery to cells.

Examples of other approaches for gene delivery to cells include electroporation, lipofection, transfection mediated by calcium phosphate, and gene transfer into tissue culture cells through the use of a method such as viral infection. In general, such transfer method involves introduction of a selection marker into cells. Next, these cells can be subjected to selection by which cells having the transgene incorporated therein and thus expressing the gene are isolated.

### (3) Second Nucleic Acid Molecule Containing a Gene Sequence Encoding the First Protein and a Sequence Which is Capable of Inducing Trans-Splicing Through Binding to Pre-Trans-Splicing Molecule

A target pre-mRNA molecule may be a molecule that is originally present within cells. In this case, a pre-trans-splicing molecule can be introduced into a cell expressing the target pre-mRNA molecule (specifically, cells expressing the first protein). Alternatively, a target pre-mRNA molecule may be a molecule that has been introduced into cells from the outside. In this case, the second nucleic acid molecule or a vector capable of expressing the nucleic acid molecule is introduced into cells in which trans-splicing can take place, wherein the second nucleic acid molecule contains a gene sequence encoding the first protein and a sequence which is capable of inducing trans-splicing through binding to pre-trans-splicing molecule.

The second nucleic acid molecule containing a gene sequence encoding the first protein and a sequence which is capable of inducing trans-splicing through binding to a nucleic acid molecule (a pre-trans-splicing molecule) preferably contains:

(a) a gene sequence encoding a first protein;

(b) a 5′ splice sequence, an Sμ sequence, and a 3′ splice sequence;

(c) a branch point; and

(d) a pyrimidine tract.

Such vector capable of expressing the nucleic acid molecule can be constructed in a manner similar to that used in the case of a pre-trans-splicing molecule.

(4) Antibody

An antibody preferable as one (first protein) of the proteins composing a fusion protein prepared by the method of the present invention will be described below. All antibodies basically have the same structure, which is a “Y”-shaped four-stranded structure (two light (polypeptide) chains and two heavy (polypepetide) chains). A light chain (or L chain) has a molecular weight of approximately 25,000 and is common among all immunoglobulins; and a heavy chain (or H chain) has a molecular weight between 50,000 and 77,000. Their structures differ depending on immunoglobulin type. A light chain and a heavy chain are linked via a disulfide bond (SS bond), so as to form a heterodimer. The heterodimers are further linked via two (right and left) disulfide bonds, so as to form a “Y”-shaped heterotetramer.

A half of the Fab region closer to the tip is extremely variable in terms of amino acid sequence so as to be able to bind to various antigens. This half of the Fab region closer to the tip is referred to as a variable region (V region). A light-chain variable region is referred to as a VL region and a heavy-chain variable region is referred to as a VH region. Fab regions other than V regions and the Fc region are relatively invariable and are referred to as constant regions (C regions). A light-chain constant region is referred to as a CL region and a heavy chain variable region is referred to as a CH region. The CH region is further divided into three (CH1 to CH3) regions. The heavy-chain Fab region comprises a VH region and CH1 and the heavy-chain Fc region comprises CH2 and CH3. A hinge part is positioned between CH1 and CH2.

The basic structure of all antibody molecules comprises 2 identical light chains (L chains) and 2 identical heavy chains (H chains), which are joined via disulfide bonds. An antibody is composed of domains comprising approximately 100 various amino acid residues. Most domains independently exist and have the same structure (immunoglobulin structure). Particularly variable domains (referred to as variable regions: a heavy chain variable region is referred to as VH; and a light chain variable region is referred to as VL) are present on the N-terminal side. Domains other than such domains are constant domains (referred to as constant regions: heavy chain constant regions are referred to as CH1, CH2, and CH3; and a light chain constant region is referred to as CL). Moreover, of these variable regions, especially variable regions are limited (referred to as complementarity-determining regions). Functions for recognizing antigens are created by varying the amino acid residues of these regions. The domain structure of an antigen molecule is a β-barrel structure comprising 9 antiparallel μ-sheets in the variable regions and 7 antiparallel β-sheets in the constant regions. Complementarity-determining regions are clustered at one end of these variable regions, so as to form antigen recognition regions.

As described above, antibodies recognize various antigens by freely varying 6 (3 from heavy chain and 3 from light chain) loop regions (complementarity-determining regions: CDRs) supported by a common framework structure (framework region: FR). Such loop structure itself cannot be completely freely varied, but can be varied to some limited extent (this structure is referred to as a canonical structure). Ability to recognize antigens is created with combination of limited structures.

H chains and L chains are encoded by different genes. Each H chain is composed of fragments (V, D, and J) responsible for variable regions and a C fragment responsible for constant regions. Each L chain is composed of fragments (V, J, and C). These gene fragments are rearranged upon antibody expression.

Examples of one (protein (1)) of the proteins forming a fusion protein that can be prepared in the present invention include an antibody H-chain variable region protein (VH); an antibody H-chain variable region protein (VH) and an antibody H chain constant region CH1 protein; an antibody L chain variable region protein (VL); and an antibody L chain variable region protein (VL) and an antibody L chain constant region CL protein.

### (5) Enzyme

A preferable enzyme that is contained in one (second protein) of the proteins forming a fusion protein that is produced by the method of the present invention will be described below. Examples of such enzyme are not particularly limited. Enzymes to be used for EIA are preferred. More specific examples of such enzyme to be used for EIA include peroxidase (PEX), alkaline phosphatase (ALP), β-galactosidase (β-Gal), malate dehydrogenase, acetylcholinesterase, and glucose oxidase.

Examples of a preferably-employed chromogenic substrate corresponding to such enzymes for EIA include: substrates for peroxidase such as TMB (3,3′,5,5′-Tetramethylbenzidine, 3,3′,5,5′-tetramethylbenzidine), OPD (o-Phenylenediamine, orthophenylene diamine), ABTS (2,2′-Amino-bis(3-ethylbenzothiazoline-6-sulfonic acid, 2,2′-azino-bis(3-ethylbenzthializoline-6-sulfonic acid); substrates for alkaline phosphatase such as 4MUP (4-methylumbelliferylphosphate) and NADP (4-nitrophenylphosphate); substrates for β-galactosidase such as 4MUG (4-methylumbelliferyl β-D-galactoside) and 2-nitrophenyl β-D-galacside; and substrates for acetylcholinesterase such as acetyl-β [methyl-thio]choline iodide.

In the present invention, of these substrates for enzyme activity determination, substrates for oxidative enzyme activity determination (in particular, substrates for peroxidase activity determination) are further preferably used in view of easy determination of enzyme activity.

The concentration of a chromogenic substrate in a substrate solution is not particularly limited. Such concentration that is preferably employed herein ranges from approximately 0.01 mg/ml to 1 mg/ml and further preferably from 0.1 mg/ml to 0.5 mg/ml in view of balance between substrate solubility and determination sensitivity.

### (6) Fusion Protein Recovery

When a fusion protein that is produced by the method of the present invention is expressed while dissolved in cells, the cells are harvested by centrifugation, suspended in an aqueous buffer, and then disrupted using an ultrasonicator or the like. Hence, a cell-free extract can be obtained. Furthermore, a fusion protein that is produced by the method of the present invention is secreted extracellularly, cell-free medium can be recovered by a method such as centrifugation. A fusion protein can be recovered and purified from such cell-free extract or medium through the use of an independent or a combination of techniques including a general protein isolation and purification method, and specifically, a solvent extraction method, a salting-out method using ammonium sulfate or the like, a desalination process, a precipitation method using an organic solvent, an anion-exchange chromatography method using a resin such as diethylaminoethyl (DEAE) sepharose, a cation exchange chromatography method using a resin such as S-Sepharose FF (produced by Pharmacia), a hydrophobic chromatography method using a resin such as butyl sepharose and phenyl sepharose, a gel filtration method using a molecular sieve, an affinity chromatography method, a chromatofocusing method, an electrophoresis method such as isoelectric focusing, and the like. Preferably, a fusion protein can be purified using an agarose carrier upon which protein G or protein L has been immobilized.

### (7) Immunoassay Method

A fusion protein obtained according to the present invention can be used for an immunoassay method. Hereinafter, the immunoassay method will be described. The immunoassay method is a method for quantitatively detecting a fine amount of a target substance contained in a sample using an enzyme-labeled antibody or antigen and an antigen-antibody reaction. An ELISA method has the following merits of: 1) allowing detection of a target substance with high sensitivity and being excellent in quantitative capability, 2) enabling measurement at a stage of crude-extraction since detection is performed using an antigen-antibody reaction and requiring no complicated steps such as purification or pretreatment, which are required by other testing methods; and 3) allowing measurement of large amounts of samples within a short time. The immunoassay method is largely classified into a sandwich method (non-competitive method) and a competitive method based on differences in measurement principle. These methods are described in detail in Enzyme immunoassay method (Eiji Ishikawa, Igaku-Shoin Ltd. (1987)) and Hypersensitive enzyme immunoassay method (Eiji Ishikawa, Japan Scientific Societies Press (1993)).

A fusion protein that is obtained according to the present invention, particularly an antibody H chain or L chain variable region protein, can be preferably used for an immunoassay method referred to as an open sandwich method. The open sandwich method is a sandwich method using an antibody VH region polypeptide and an antibody VL region polypeptide, as disclosed in JP Patent No. 378411 and WO2006/033413. The open sandwich method makes it possible to measure low-molecular-weight compounds, which have been difficult to measure with the use of conventional sandwich methods. Specifically, such open sandwich method involves preparing a VH region polypeptide and a VL region polypeptide of an antibody that specifically recognizes an antigen, labeling one of the polypeptides with a reporter molecule to prepare a labeled polypeptide, immobilizing the other polypeptide on a solid phase to prepare an immobilized polypeptide, causing an antigen-containing sample and the labeled polypeptide to come into contact with the solid phase, and measuring the amount of the reporter molecule of the labeled polypeptide bound to the immobilized polypeptide. Based on such open sandwich method, a method for measuring the concentration of an antigen (first method) wherein a reporter molecule is an enzyme or a fluorescent dye is provided.

In a preferred embodiment of the first method, such polypeptide (to be immobilized) is immobilized on a solid phase via binding of biotin or a tag sequence with avidin or streptavidin. In another preferred embodiment of the first method, an enzyme that is a reporter molecule is Escherichia coli alkaline phosphatase or a variant thereof and a fluorescent dye is fluorescein or a derivative thereof.

Furthermore, according to the present invention, a method for measuring the concentration of an antigen (second method) is provided as a second means, which comprises preparing a VH region polypeptide and a VL region polypeptide of an antibody that specifically recognizes an antigen, labeling the VH region polypeptide with a 1^{st} reporter molecule, labeling the VL region polypeptide with a 2^{nd} reporter molecule, mixing a sample containing the antigen, the labeled VH region polypeptide, and the labeled VL region polypeptide, and measuring changes resulting from the interaction between the 1^{st} reporter molecule and the 2^{nd} reporter molecule.

In one embodiment of the 2^{nd} method, the 1^{st} reporter molecule and the 2^{nd} reporter molecule are different types of fluorescent dye and the amounts of complexes are measured based on the amount of fluorescence generated as a result of energy transfer between the two reporter molecules. In this case, such different types of fluorescent dye are preferably fluorescein or a derivative thereof and rhodamine or a derivative thereof.

Hereafter, the invention will be further described in detail with reference to the following example, but the present invention is not limited to such example.

### EXAMPLE

### Example

### Preparation of a Fusion Protein Through Trans-Splicing Using a Vector Containing an Antibody Variable Region Sequence and a Vector Containing an Alkaline Phosphatase Sequence

### (1) Materials and Methods

### Cultured Cells

Monkey-kidney-derived COS-1 cells were used as cells for expressing a model system. Cells were cultured in DMEM medium supplemented with 10% fetal calf serum, penicillin and streptomycin in a humidified incubator at 37° C. and 5% CO2.

Furthermore, J558L cells expressing the λ chain of an anti-4-hydroxy-3-nitrophenyl acetyl (NP) antibody to be used for enzyme immunoassay (ELISA) were purchased from the European Collection of Cell Cultures.

### Primers

Mun2EcoFor;

(SEQ ID NO: 1)

GGAATTCTTGTTGTTAACTTGTTTATTGC
SeapHis6p2;

(SEQ ID NO: 2)

CCGGGTTACTCTAGAGTCGGGGCGGCCGGCCACCACCACCACCACCACTG
ATAAGATACATTGATGAG
PSEAPFor;

(SEQ ID NO: 3)

GGAATTCCATGGCTTAATTAAGGCGCGCCTCCGGAATCATCCCAGTTGAG
GAGGAGAA
PSEAPBk;

(SEQ ID NO: 4)

CCGGAATTCATATGGGAAGCGGTCCATTGCCAGGGGTAT
IgMInt5;

(SEQ ID NO: 5)

GGAATTCTTAATTAACTTAAGTAGGTTTGGGGGATG
IgMInt3;

(SEQ ID NO: 6)

GGAATTCTCCGGAACCTGCAGTCAAGAGAACAC
VlamMfeBack;

(SEQ ID NO: 7)

CAGGTCCAATTGGATGCTGTTGTGACTCAGGAATC
VHAflfor2;

(SEQ ID NO: 8)

TTTAAGCTTAAGGACTCACCCGAGGAACTGTGAGAGTGGT
SuEcoFor;

(SEQ ID NO: 9)

CCAGTACAGCTCAGTCTAGCACATCTGAATTCAGCTCAGCCCC
SuAflBack1;

(SEQ ID NO: 10)

CCGAGGTGAGTGTGAGAGGACAGGGGCTTAAGTATGGATACGCAGAAGGA
AG
SuAflBack2;

(SEQ ID NO: 11)

GGTCGGCTGGACTAACTCTCCAGCCACCTTAAGGACCCAGACAGAGAAAG
CC
Int20028F;

(SEQ ID NO: 12)

GTTTCGTCCTGTATACCAGG
IntEcoNcoB;

(SEQ ID NO: 13)

GGAATTCCATGGCTGAGGACCAGAGAGGGATAAAAG
NPVHMfeBack;

(SEQ ID NO: 14)

CAGGTCCAATTGCAGCAGCCTGGG
MunIgMCH2for;

(SEQ ID NO: 15)

CACATTTACATTGGGATTCAT

### Vectors

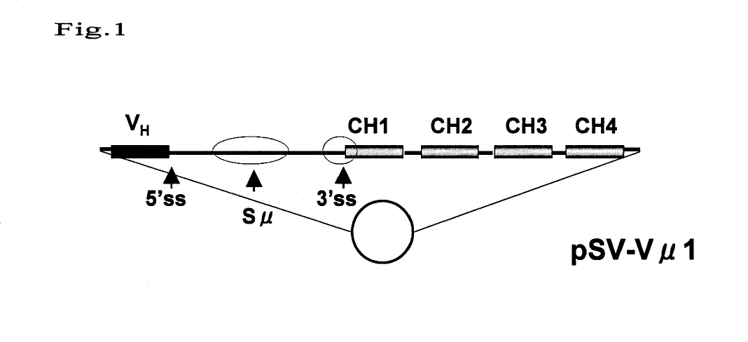

An anti-4-hydroxy-3-nitrophenylacetyl (NP) antibody heavy chain was used as a target antibody model. A pSV-Vμ1 vector capable of expressing the model in eukaryotic cells was used. pSV-Vμ1 used herein had been provided by Dr. Michael Neuberger at the Medical Research Council U.K. (Reference: EMBO, Vol. 2, 1983, pp. 1373-1378) (FIG. 1).

A trans-splicing vector (hereinafter, TS vector) was constructed by the following procedure.

First, the following steps were conducted for introducing an His-tag into the C-terminus of secretory human placenta alkaline phosphatase (SEAP) encoded by pSEAP2_control (Clontech, Inc.).

A sequence around the 3′ end of an SEAP sequence containing an His-tag was amplified by PCR reaction (condition 1 (Table 1)) using pSEAP2_control as a template, an Mun2EcoFor primer, and a SeapHis6p2 primer. The amplified product was digested with restriction enzymes EcoR I and Xba I. The digested product was inserted into pSEAP2_control that had been digested with restriction enzymes Xba I and Mun I. The nucleotide sequence of the product was confirmed (hereinafter, pSEAP-His).

Next, an antibody variable region gene and an IgM-derived intron were inserted into the 5′ N-terminal side of the SEAP gene by the following procedure.

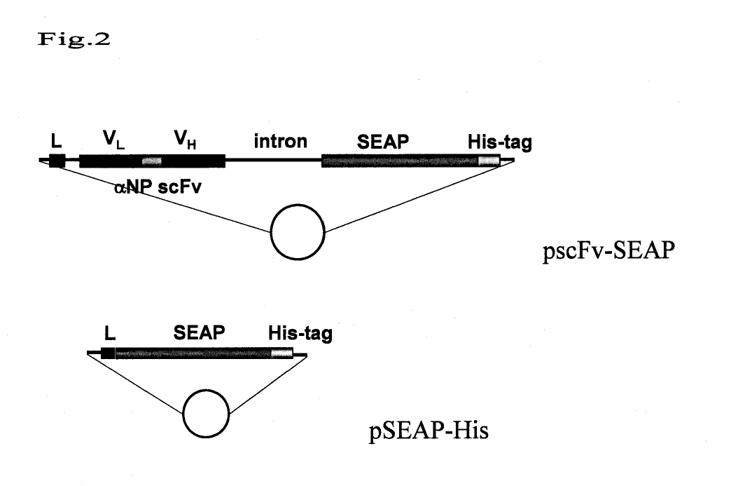

A sequence around the N-terminus of SEAP was amplified by PCR (condition 2 (Table 2)) using pSEAP2_control as a template, a pSEAPFor primer, and a pSEAPBk primer. The thus amplified product was treated with a restriction enzyme EcoR I and then incorporated into the EcoR I site of pUC-19 (TaKaRa Bio, Co.). To the upstream thereof, an intron sequence between CH1 and CH2 regions, which had been amplified (condition 3 (Table 3)) from pSV-Vμ1 using an IgMInt5 primer and an IgMInt3 primer, was inserted using Pac I and BspE I sites. Furthermore, to the upstream thereof, a sequence of an anti-NP single-chain antibody (scFv), which had been amplified by PCR (condition 4 (Table 4)) using pGEMSCA (Suzuki et al, J. Biochem., 122, 322-329 (1997)) encoding the anti-NP single-chain antibody (scFv) as a template, a VlamMfeBack primer and a VHAflFor2 primer, was incorporated. Thus, a pUC-scFv-int-SEAP plasmid was constructed. pUC-scFv-int-SEAPn was treated with restriction enzymes EcoR I and Nde I, so as to excise a DNA fragment having the N-terminal sequence of scFv-intron-SEAP. The DNA fragment was then incorporated into pSEAP-His that had also been treated with EcoR I and Nde I, so that a vector (hereinafter, pscFv-SEAP) was constructed (FIG. 2).

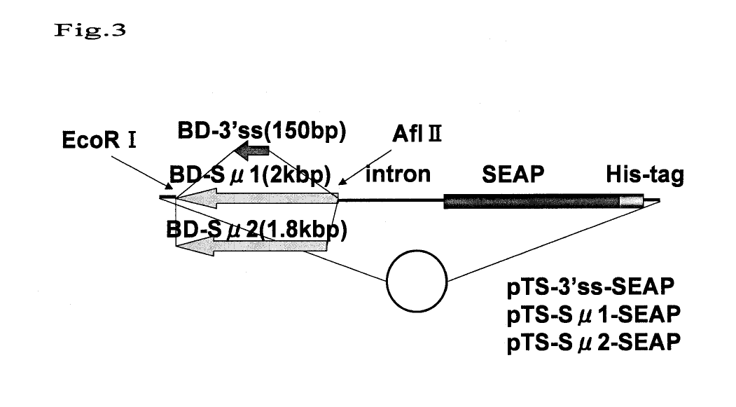

Next, the scFv sequence of pscFvSEAP prepared as described above was substituted with a sequence (hereinafter, BD) capable of hybridizing to a target sequence, so that a target TS vector was constructed. Specifically, as sequences to which BD hybridizes, a repeat sequence (Sμ sequence) involved in antibody class switch and common to IgM and a 3′ splice site (3′ ss) were selected. For amplification of BD, PCR reaction (condition 1 (Table 1)) was performed using pSV-Vμ1 as a template, an SmuEcoFor primer and an SmuAflBack1 primer (condition 5 (Table 5)), or an SmuEcoFor primer and an SmuAflBack2 primer (condition 6 (Table 6)), or an Int20028F primer and an IntEcoNcoB primer (condition 7 (Table 7)). These BDs were designated Sμ1, Sμ2, and 3′ ss, respectively, and then treated with restriction enzymes EcoR I and Afl II. The products were inserted into pscFv-SEAP that had also been treated with restriction enzymes EcoR I and Afl II. Thus, 3 types of TS vector (pTS-3′ss-SEAP, pTS-Sμ1-SEAP, and pTS-Sμ2-SEAP) were constructed (FIG. 3).

TABLE 1
Condition 1 (25 cycles of steps 2 to 4)

| Temperature | Time | |
|---|---|---|
| 94° C. | 2 | min |
| 94° C. | 30 | sec |
| 55° C. | 30 | sec |
| 72° C. | | |
| 72° C. | 5 | min |
| 16° C. | | |

TABLE 2
Condition 2 (25 cycles of steps 2 to 4)

| Temperature | Time | |
|---|---|---|
| 94° C. | 2 | min |
| 94° C. | 30 | sec |
| 55° C. | 30 | sec |
| 72° C. | 1 | min |
| 72° C. | 5 | min |
| 16° C. | | |

TABLE 3
Condition 3 (25 cycles of steps 2 to 4)

| Temperature | Time | |
|---|---|---|
| 94° C. | 2 | min |
| 94° C. | 30 | sec |
| 55° C. | 30 | sec |
| 72° C. | 1 | min |
| 72° C. | 5 | min |
| 16° C. | | |

TABLE 4
Condition 4 (25 cycles of steps 2 to 4)

| Temperature | Time |
|---|---|
| 94° C. | 2 min |
| 94° C. | 30 sec |
| 55° C. | 30 sec |
| 72° C. | 1 min 30 sec |
| 72° C. | 5 min |
| 16° C. | |

TABLE 5
Condition 5 (20 cycles of steps 2 to 3)

| Temperature | Time |
|---|---|
| 94° C. | 5 min |
| 94° C. | 15 sec |
| 72° C. | 3 min 30 sec |
| 72° C. | 5 min |
| 16° C. | |

TABLE 6
Condition 6 (20 cycles of steps 2 to 3)

| Temperature | Time |
|---|---|
| 94° C. | 5 min |
| 94° C. | 15 sec |
| 72° C. | 3 min 30 sec |
| 72° C. | 5 min |
| 16° C. | |

TABLE 7
Condition 7 (25 cycles of steps 2 to 4)

| | |
|---|---|
| Temperature | Time |

| | | |
|---|---|---|
| 94° C. | 5 | min |
| 94° C. | 15 | sec |
| 55° C. | 30 | sec |
| 72° C. | 40 | sec |
| 72° C. | 5 | min |
| 16° C. | | |

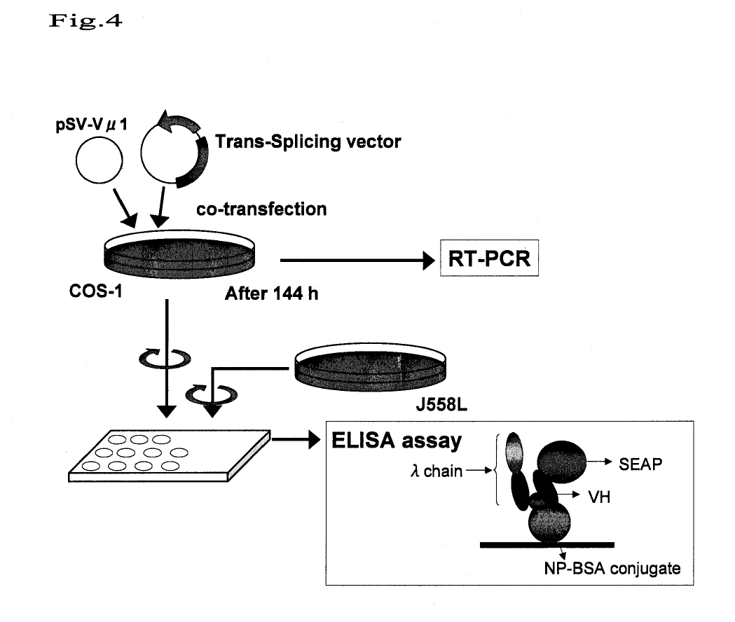

### Transfection (FIG. 4)

COS-1 cells (1.5×10⁵ cells) were seeded on a 35-mm dish (IWAKI) for adhesion cell culture. 12 to 24 hours later, transfection was performed according to general protocols using a pSV-Vμ1 vector and one of the 3 above-constructed types of TS vector (pTS-3′ ss-SEAP, pTS-Sμ1-SEAP, and pTS-Sμ2-SEAP), and a transfection reagent, Lipofectamine 2000 (Invitrogen, Inc.) or COSFectin (BIO RAD, Co.).

### Reverse Transcription Polymerase Chain Reaction (RT-PCR) (FIG. 4)

To confirm mRNA generated through trans-splicing, total RNA was extracted from the cells at 48 hours after transfection using an RNAspin Mini (GE Healthcare). Exscript (TAKARA Bio, Co.) was used for the extract, so as to prepare cDNA. Amplification (condition 8 (Table 8)) was performed by PCR reaction using the cDNA as a template, and using an NPVHMfeBack primer and a pSEAPBk, or NPVHMfeBack primer and a CH2for primer.

TABLE 8
Condition 8 (35 cycles of steps 2 to 4)

| Temperature | Time |
|---|---|
| 94° C. | 5 min |
| 94° C. | 30 sec |
| 60° C. | 30 sec |
| 72° C. | 1 min 30 sec |
| 72° C. | 10 min |
| 16° C. | |

In the former reaction, it was expected that mRNA (generated as a result of trans-splicing) encoding the anti-NP antibody heavy chain variable region (VH) and an SEPA fusion protein would be detected. In the latter reaction, it was expected that mRNA (generated as a result of cis-splicing) encoding the anti-NP antibody heavy chain variable region (VH) and a CH1 region-CH2 region fusion protein would be detected. After PCR reaction, each reaction solution was electrophoresed using 1.5% agarose gel and then the position and amount of the thus generated band were confirmed.

### Enzyme Immunoassay (ELISA) (FIG. 4)

An antigen (NP-BSA conjugate) was prepared with PBS at a concentration of 100 μg/ml. The antigen solution was dispensed at 100 μl per well of a 96-well white plate (3922, Corning, Inc.) and then stored at 4° C. overnight for immobilization. After the plate had been washed, 200 μl each of immunoblock diluted 5 folds (Dainippon Sumitomo Pharma Co., Ltd.) was added and then incubation was performed for 2 hours at room temperature for blocking. To the solution, 30 μl of the culture supernatant at 144 hours after transfection using COSFectin and 10 μl of the culture supernatant of J558L cells expressing the anti-NP antibody λ chain were added, followed by 1 hour of incubation at room temperature. After washing, 100 μl of an endogenous AP inhibition solution in a Protein Assay Kit-SEAP—(TOYOBO) was added, followed by 30 minutes of incubation at 37° C. After another washing, 100 μl of a luminescent substrate in the same kit was added. The amount of luminescence accumulated over 10 seconds was calculated and then measured using a Luminescenser-JNR AB2100 (ATTO, Co.).

### (2) Results

### RT-PCR

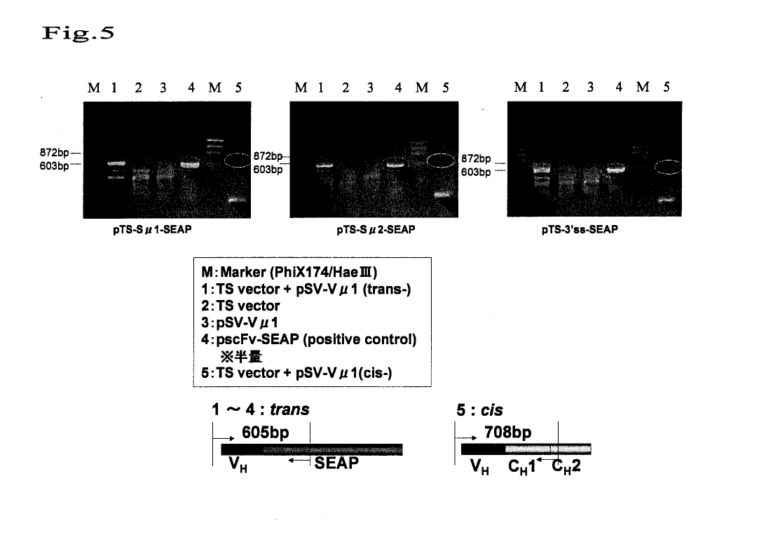

Bands at around a target size of 605 bp could be confirmed in all the COS-1 cells into which PSV-Vμ1 and any one of the 3 types of TS vector had been introduced. Moreover, the thickness of each band was almost a half of that in the case of cells in which positive control pScFv-SEAP had been introduced and was significantly thicker than the bands derived from cis-splicing products. On the other hand, no such bands (at around 605 bp) were observed in the cases of cells into which the TS vector alone or PSV-Vμ1 alone had been introduced. These results suggested that mRNA had been generated by trans-splicing at high efficiencies between the pre-mRNA of the target and the pre-mRNA of the TS vector (FIG. 5).

### ELISA

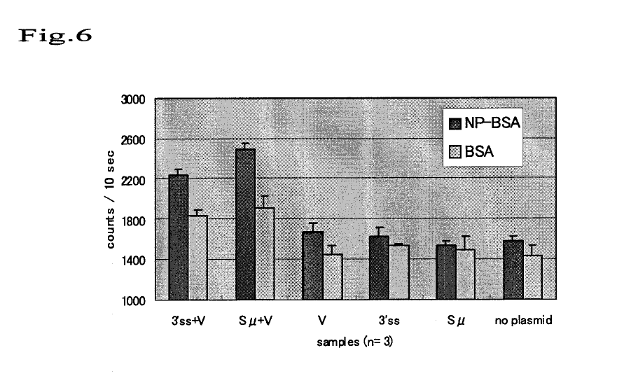

AP activity of the culture supernatant of COS-1 cells into which both the target (pSV-Vμ1) and the TS vector (3′ ss or Sμ-1) had been introduced was determined in the presence of the culture supernatant of J588L. AP activity was detected at significantly higher levels than those in the culture supernatant of cells into which none or only one of the vectors had been introduced. Furthermore, AP activity was compared based on the presence or the absence of an antigen (comparison between a case in which NP-BSA had been immobilized and a case in which BSA had been immobilized). A significantly higher AP activity level was confirmed in the case in which NP-BSA had been immobilized compared with the case in which BSA had been immobilized (FIG. 6). As described above, a VH-SEAP fusion protein derived from RNA generated by TS had been secreted in these culture supernatants. Hence, exertion of antigen-binding ability in the presence of the λ chain was strongly suggested.

1. A method for producing a fusion protein comprising a first protein and a second protein, which comprises the steps of:
introducing a nucleic acid molecule (pre-trans-splicing molecule) or a vector capable of expressing such a nucleic acid molecule into a cell expressing the first protein, wherein the nucleic acid molecule contains a gene sequence encoding the second protein and a sequence which is capable of inducing trans-splicing through binding to pre-mRNA that is generated from a region containing a gene sequence that encodes the first protein; and recovering a fusion protein comprising the first protein and the second protein generated within the cell.

2. A method for producing a fusion protein comprising a first protein and a second protein, which comprises the steps of:
introducing a first nucleic acid molecule containing a gene sequence that encodes the first protein or a vector capable of expressing the nucleic acid molecule and a second nucleic acid molecule (pre-trans-splicing molecule) or a vector capable of expressing the nucleic acid molecule into a cell in which trans-splicing can take place, wherein the second nucleic acid molecule contains a gene sequence encoding the second protein and a sequence which is capable of inducing trans-splicing through binding to pre-mRNA that is generated from a region containing a gene sequence that encodes the first protein; and
recovering a fusion protein comprising the first protein and the second protein generated within the cell.

3. The method according to claim 1, wherein the nucleic acid molecule (pre-trans-splicing molecule) containing a gene sequence encoding the second protein and a sequence which is capable of inducing trans-splicing through binding to pre-mRNA that is generated from a region containing a gene sequence that encodes the first protein is a nucleic acid molecule containing at least one sequence selected from among a 5′ splice sequence, a 3′ splice sequence, and an Sμ sequence or a vector capable of expressing the nucleic acid molecule.

4. The method according to claim 1, wherein the nucleic acid molecule (pre-trans-splicing molecule) containing a gene sequence encoding the second protein and a sequence which is capable of inducing trans-splicing through binding to pre-mRNA that is generated from a region containing a gene sequence that encodes the first protein is a nucleic acid molecule containing:
(a) at least one sequence selected from among a 5′ splice sequence, a 3′ splice sequence, and an Sμ sequence;
(b) a branch point; and
(c) a pyrimidine tract;
or is a vector capable of expressing the nucleic acid molecule.

5. The method according to claim 1, wherein the nucleic acid molecule (pre-trans-splicing molecule) containing a gene sequence encoding the second protein and a sequence which is capable of inducing trans-splicing through binding to pre-mRNA that is generated from a region containing a gene sequence that encodes the first protein is a nucleic acid molecule encoded by a plasmid vector or a retrovirus vector.

6. The method according to claim 1, wherein the first protein is a protein containing an antibody H chain variable region protein (VH).

7. The method according to claim 1, wherein the first protein is a protein containing the antibody H chain variable region protein (VH) and the second protein is a protein containing an antibody H chain constant region CH1 protein.

8. The method according to claim 1, wherein the first protein is a protein containing an antibody L chain variable region protein (VL).

9. The method according to claim 1, wherein the first protein is a protein containing the antibody L chain variable region protein (VL) and the second protein is a protein containing an antibody L chain constant region CL protein.

10. The method according to claim 1, wherein the cell expressing the first protein is a cell producing an antibody.

11. The method according to claim 1, wherein the cell expressing the first protein is a hybridoma.

12. The method according to claim 1, wherein the cell expressing the first protein is a DT40 chicken somatic cell.

13. The method according to claim 1, wherein the second protein is a protein containing an enzyme.

14. The method according to claim 1, wherein the second protein is a protein containing alkaline phosphatase, peroxidase, β-galactosidase, or luciferase.

15. An immunoassay method, comprising the steps of: producing a fusion protein by the method according to claim 1; and performing immunoassay using the thus obtained fusion protein.

16. An immunoassay method, comprising the steps of: producing a fusion protein containing an antibody H chain variable region protein (VH) and/or an antibody L chain variable region protein (VL) by the method according to claim 1; and performing immunoassay using the thus obtained fusion protein.


SEQUENCE LISTING (15 sequences)

SEQ ID NO: 1 - DNA, 29 bp, Artificial
  Description of Artificial Sequence Synthetic DNA
ggaattcttgttgttaacttgtttattgc

SEQ ID NO: 2 - DNA, 68 bp, Artificial
  Description of Artificial Sequence Synthetic DNA
ccgggttactctagagtcggggcggccggccaccaccaccaccaccactgataagatacattgatgag

SEQ ID NO: 3 - DNA, 58 bp, Artificial
  Description of Artificial Sequence Synthetic DNA
ggaattccatggcttaattaaggcgcgcctccggaatcatcccagttgaggaggagaa

SEQ ID NO: 4 - DNA, 39 bp, Artificial
  Description of Artificial Sequence Synthetic DNA
ccggaattcatatgggaagcggtccattgccaggggtat

SEQ ID NO: 5 - DNA, 36 bp, Artificial
  Description of Artificial Sequence Synthetic DNA
ggaattcttaattaacttaagtaggtttgggggatg

SEQ ID NO: 6 - DNA, 33 bp, Artificial
  Description of Artificial Sequence Synthetic DNA
ggaattctccggaacctgcagtcaagagaacac

SEQ ID NO: 7 - DNA, 35 bp, Artificial
  Description of Artificial Sequence Synthetic DNA
caggtccaattggatgctgttgtgactcaggaatc

SEQ ID NO: 8 - DNA, 40 bp, Artificial
  Description of Artificial Sequence Synthetic DNA
tttaagcttaaggactcacccgaggaactgtgagagtggt

SEQ ID NO: 9 - DNA, 43 bp, Artificial
  Description of Artificial Sequence Synthetic DNA
ccagtacagctcagtctagcacatctgaattcagctcagcccc

SEQ ID NO: 10 - DNA, 52 bp, Artificial
  Description of Artificial Sequence Synthetic DNA
ccgaggtgagtgtgagaggacaggggcttaagtatggatacgcagaaggaag

SEQ ID NO: 11 - DNA, 52 bp, Artificial
  Description of Artificial Sequence Synthetic DNA
ggtcggctggactaactctccagccaccttaaggacccagacagagaaagcc

SEQ ID NO: 12 - DNA, 20 bp, Artificial
  Description of Artificial Sequence Synthetic DNA
gtttcgtcctgtataccagg

SEQ ID NO: 13 - DNA, 36 bp, Artificial
  Description of Artificial Sequence Synthetic DNA
ggaattccatggctgaggaccagagagggataaaag

SEQ ID NO: 14 - DNA, 24 bp, Artificial
  Description of Artificial Sequence Synthetic DNA
caggtccaattgcagcagcctggg

SEQ ID NO: 15 - DNA, 21 bp, Artificial
  Description of Artificial Sequence Synthetic DNA
cacatttacattgggattcat